SEQUENCE LISTING (2 sequences)

SEQ ID NO: 1 - DNA, 47 bp, Artificial Sequence
  Sequence generated by inventor for exemplifying invention.
taacttgacctgagctagtagagctataggcgatagccctctaagcc

SEQ ID NO: 2 - DNA, 47 bp, Artificial Sequence
  Sequence generated by inventor for exemplifying invention.
attgaactggactcgatcatctcgatatccgctatcgggagattcgg

(19) United States
(12) Patent Application Publication
(10) Pub. No.: US 2005/0186576 A1
(43) Pub. Date: Aug. 25, 2005

(54) Polymer sequencing using selectively labeled monomers and data integration

(75) Inventors: Selena Chan, San Jose, CA (US); Xing Su, Cupertino, CA (US); Andrew A. Berlin, San Jose, CA (US); Tae-Woong Koo, San Francisco, CA (US)

Correspondence Address: Julia A. Hodge;c/o BLAKELY, SOKOLOFF, TAYLOR & ZAFMAN LLP, Seventh Floor, 12400 Wilshire Boulevard, Los Angeles, CA, 90025, US

(73) Assignee: INTEL CORPORATION, Santa Clara, CA (US)

(21) Appl. No.: 10/782,014

(22) Filed: Feb. 19, 2004

(51) Int. Cl. C12Q 1/68; G01N 33/48; G01N 33/50

(52) U.S. Cl. 435/6; 702/20

(57) ABSTRACT

Methods and apparatuses for sequencing single polymer molecules, such as a nucleic acid strand, are discussed. A discussed method comprises dividing a polymer sample into a number of polymer subsamples equal to the number of different monomer types and partially labeling only one of the monomer types in each polymer subsample. The method may further comprise placing a subsample into a reaction chamber, sequentially separating each monomer from the polymer subsample, and detecting the labels of each separated labeled monomer as a function of time. The time between each labeled monomer may be used to construct a monomer-time map for each polymer sub-sample using overlapping data analysis and frequency analysis. Time maps may then be assembled/aligned into a polymer sequence from the monomer-time maps of each of the polymer subsamples using non-overlapping data analysis.

### TECHNICAL FIELD

The disclosed methods and devices relate to the fields of molecular biology and genomics. More particularly, the disclosed methods and apparatus realte to polymer sequencing, including nucleic acids such as deoxyribonucleic acid (DNA) and ribonucleic acid (RNA). The disclosed methods can be used in biochemical research and various medical or clinical applications.

### BACKGROUND

Genetic information is stored in the form of very long nucleic acid molecules such as deoxyribonucleic acid (DNA) and ribonucleic acid (RNA). The human genome contains approximately three billion nucleotides of DNA sequence. DNA sequence information can be used to determine multiple characteristics of an individual as well as and many common diseases, such as cancer, cystic fibrosis and sickle cell anemia. Determination of the entire three billion nucleotide sequence of the human genome has provided a foundation for identifying the genetic basis of such diseases.

Traditionally, polynucleic acids, have been sequenced by one of two major approaches: 1) Chemical degradation and fragment sizing by gel electrophoresis or 2) dideoxy fragment matching by hybridization (see Sanger et al. in Molecular Cloning: A Laboratory Manual. Cold Spring Harbor Laboratory Press, N.Y., Vol. 1-3 (1989) and D. Glover, DNA Cloning Volume I: A Practical Approach. IRL Press, Oxford, 1985).

In the“fragment sizing” approach, DNA samples are stripped down to a single strand and exposed to a chemical that destroys one of the four nucleotides, adenine (A), thymine (T), guanine (G), or cytosine (C). For example, if A is destroyed, the strand of DNA will be digested into various labeled nucleic acid fragments that ended in A. This procedure is repeated for the other three types of nucleotides. The fragments are then sized (sorted according to length) by gel electrophoresis. The various lengths of the fragments show the times from the labeled end to the known type of nucleotide. If there are no gaps in coverage, the original DNA strand sequence can be determined from these fragment sequences.

The fragment sizing approach has several disadvantages, including that some regions and longer fragments of DNA are hard to sequence because of DNA's secondary structure and there may be only small differences in mobility between fragments, even between fragments of significantly different lengths. Generally, this fragment size limitation ranges up to from about 0.5 to about 1 kilobases (kbs) without significantly decreasing the resolution and accuracy of this technique. This is much shorter than the length of the functional unit of DNA, referred to as a gene, which can be 10 to 100,000 or more nucleotides in length. In :fragment sizing,” determination of a complete gene sequence requires that many copies of the gene be produced, cut into overlapping fragments and sequenced. Then, the overlapping DNA sequences may be assembled into the complete gene. The fragment sizing method is also very time consuming and does not work well for sequencing the genomes of complex organisms, such as humans. In addition, the preparation work before and analysis after electrophoresis is inherently expensive and slow.

The “fragment matching” approach is generally disclosed in U.S. Pat. No. 5,653,939. This method typically employs an array of test sites attached to a substrate. Each test site either includes a) “probe” molecules which are adapted to bond or hybridize with a predetermined target nucleic acid sequence or b) the unknown target nucleic acid fragments which are then exposed to the probe molecules. The bonding of a particular nucleic acid sequence with a probe molecule at a test site changes the electrical, mechanical, and/or optical properties of each test site. When an electrical, mechanical, or optical signal is then applied to these test sites, the change in properties can be detected and measured to determine which probes have bonded with their respective target nucleic acid sequence. Applying this method to smaller nucleic acid fragments allows one to map the entire sequence of both the fragments and the nucleic acid from which they were derived.

However, the fragment matching method is not well suited for identifying long nucleic acid sequences. The problem with the fragment matching approach is that it has a relatively low accuracy due to mis-hybridization and interference by repetitive or redundant sequences. Another problem with this method is that the materials needed for sequencing by this method are complicated to manufacture. Therefore, a need exists for a faster, consistent, and more economical means to sequence DNA and other polymers.

Recently, several research groups have developed the capability to directly detect and identify single fluorescent molecules in solution, including fluorescently labeled nucleotides that are either intact or cleaved from strands of DNA (see R. A. Keller, et al., Applied Spectroscopy, 50(7): 12A-32A (1996); W. P. Ambrose et al,. Chem. Rev., 99: 2929-2956 (1999)). The problem with direct detection of intact DNA for sequencing is that the distance between two adjacent nucleotides in a DNA chain is too small (ca. 0.34 nm) to currently be measured directly. Similarly, the problem with sequencing DNA by detecting individual nucleotides is that it requires the labeling of all of the nucleotides in a particular strand. In reality, this is extremetly difficult to accomplish. Both of these methods also have the problem of misidentification of the nucleotide if the label or dye is defective in a particular nucleotide position.

### BRIEF DESCRIPTION OF THE DRAWINGS

In order that the disclosed methods and devices may be better understood, several embodiments thereof will now be described by way of example only and with reference to the accompanying drawings, wherein,

FIG. 1 depicts an exemplary apparatus 100 (not to scale) and scheme as to how individual randomly labeled nucleotides of DNA 110 are sequentially, selectively cleaved with an exonuclease so that each nucleotide can be detected as a function of their time since the detection of the first cleaved nucleotide. In FIG. 1, single molecule optical fluorescence spectroscopy is depicted as one possible means of detection,

FIG. 2 depicts a partial labeling of adenosine in a DNA subsample in accordance with one embodiment of the disclosed methods and devices and an exemplary method for constructing a nucleotide time map 310, 320, 330, 340 for one type of labeled nucleotide 220, based on measured times between labeled nucleotides 220 in a number of complementary nucleic acid strands 230, 240, 250. The times between labeled nucleotides 220 may be compiled into a time map 310, 320, 330, 340 for each type of nucleotides labeled as described herein. Distances between the labelled nucleotides 220 may then be calculated from these time maps 310, 320, 330, 340. The sequence 210 of the complementary strand 230, 240, 250 is shown, along with exemplary locations for labeled nucleotides 220. As indicated 260, where identical nucleotides are located adjacent to each other, this will be detected as an increased frequency of labeling at that location;

FIG. 3 depicts how a complementary DNA sequence 210 may be assembled by aligning the four nucleotide time separation maps 310, 320, 330, 340 according to the non-overlapping rules. The template nucleic acid 200 will be an exact complement of the determined sequence 210. Computerized and statistical tools can assist in this process.

FIG. 4 depicts an exemplary method for constructing 450 time maps 310, 320, 330, 340 for labeled nucleotides 220.

FIG. 5 illustrates an exemplary method for aligning 520 time maps 310, 320, 330, 340 to obtain a nucleic acid sequence 200 of the complementary strand 210.

### DETAILED DESCRIPTION OF THE PRESENTLY PREFERRED EMBODIMENTS

The presently disclosed methods and devices solve these problems by not requiring direct detection of each nucleotide in a sequence in a particular DNA strand but rather detecting randomly labeled nucleotides of a particular nucleotide type from multiple strands such that every nucleotide of a particular type is eventually detected. The disclosed method can be extended to other types of polymers. The disclosed methods also solve the problem of incomplete labeling and misdetection of nucleotides or monomers by statistically sampling nucleotide or monomer types detected over multiple runs. In this way, statistically, every nucleotide or monomer in the sequence will be detected irrespective of whether sometimes a particular nucleotide or monomer is unlabeled or undetected. Using the disclosed methods, longer polymer molecules can be sequenced and more accurate data can be obtained than with other single molecule detection methods because assembling specific fragment data is not required. In addition, it is potentially more sensitive and cost effective because the method is based on a smaller subset of single molecule detections than either the fragment sizing or fragment matching approaches.

Definitions

For the purposes of the present disclosure, the following terms have the following meanings.

“Antibody” includes polyclonal and monoclonal antibodies as well as fragments thereof. Antibodies also include recombinant antibodies, chemically modified antibodies and humanized antibodies, all of which can be single-chain or multiple-chain.

“Nucleic acid” means either DNA or RNA, single-stranded, double-stranded or triple stranded, as well as any modified form or analog of DNA or RNA. A “nucleic acid” may be of almost any length, from 10 to 5,000,000 or more bases in length, up to a full-length chromosomal DNA molecule.

“Nucleotide precursor” refers to a nucleotide before it has been incorporated into a nucleic acid. In some embodiments of the disclosed methods and devices, the nucleotide precursors are ribonucleoside triphosphates or deoxyribonucleoside triphosphates. It is contemplated that various substitutions or modifications may be made in the structure of the nucleotide precursors, so long as they are still capable of being incorporated into a complementary strand by a polymerase. For example, in certain embodiments the ribose or deoxyribose moiety may be substituted with another pentose sugar or a pentose sugar analog. In other embodiments, the phosphate groups may be substituted, such as by phosphonates, sulphates or sulfonates. In still other embodiments, the purine or pyrimidine bases may be modified or substituted by other purines or pyrimidines or analogs thereof, so long as the sequence 210 of nucleotide precursors incorporated into the complementary strand 230, 240, 250 reflects the sequence of a template strand 200.

“Tags” or “labels” are used interchangeably to refer to any atom, molecule, compound or composition that can be used to identify a nucleotide 220 to which the label is attached. In various embodiments of the disclosed methods and devices, such attachment may be either covalent or non-covalent. In non-limiting examples, labels may be fluorescent, phosphorescent, luminescent, electroluminescent, chemiluminescent or any bulky group or may exhibit Raman or other spectroscopic characteristics. It is anticipated that virtually any technique capable of detecting and identifying a labeled nucleotide 220 may be used, including visible light, ultraviolet and infrared spectroscopy, Raman spectroscopy, nuclear magnetic resonance, positron emission tomography, scanning probe microscopy and other methods known in the art. In certain embodiments, nucleotide precursors may be secondarily labeled with bulky groups after synthesis of a complementary strand 230, 240, 250 but before detection of labeled nucleotides 220.

The terms “a” or “an” entity may refer to one or more than one of that entity.

As used herein, “operably coupled” means that there is a functional interaction between two or more units of an apparatus 100 and/or system. For example, a detector may be “operably coupled” to a computer if the computer can obtain, process, store and/or transmit data on signals detected by the detector.

The disclosed method, compositions, and device are of use in sequencing polymers. One disclosed method of sequencing a polymer comprises generally:

a) dividing a polymer sample into a number of polymer subsamples equal to the number of different monomer types comprising the polymer sample, wherein only one of the monomer types in each polymer subsample is partially labeled such that the average time between two adjacent labeled monomers is significantly larger than the average time between two adjacent monomers of the same type in the polymer subsample before labelling;

b) sequentially separating each monomer from the polymer subsample or;

c) detecting the labels of each separated labeled monomer as a function of time;

d) assembling a monomer-time map for each polymer sub-sample; and

e) assembling a polymer sequence from the monomer-time maps of each of the polymer subsamples.

The polymer divided in the disclosed methods and devices include any covalent molecular arrangement of monomers. Examples of polymers divided in the disclosed methods and devices include, but are not limited to, nucleic acids such as DNA and RNA, proteins, carbohydrates and other oligosaccharides, plastics, resins, and the like. For ease of illustration, nucleic acids will be used to exemplify the disclosed methods and devices. However, the disclosed methods and devices is not limited to this example. In certain embodiments, the methods and device are suitable for obtaining sequences of very long polymer molecules.

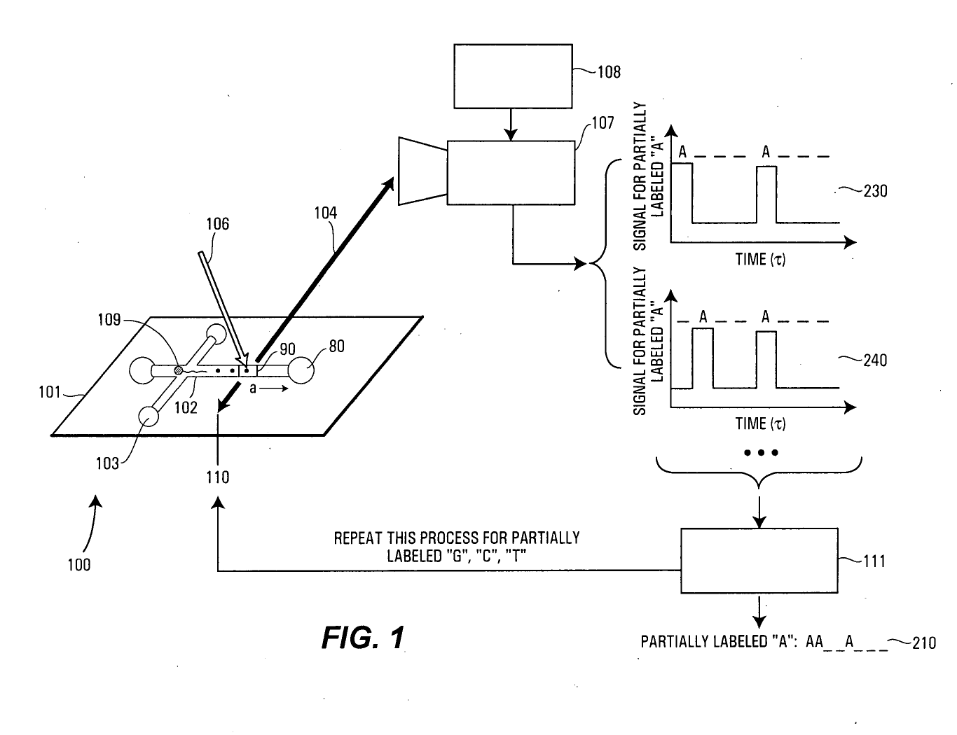

According to one embodiment, the polymer sample is divided into a number of polymer subsamples equal to the number of different monomer types comprising the polymer sample. For example, one embodiment of the disclosed methods and devices relates to nucleic acid sequencing and is illustrated in FIGS. 1-3. DNA is a nucleic acid comprised of four nucleotide monomers, adenine (A), cytosine (C), guanine (G), and cytosine (C). Therefore, the initial DNA polymer sample would be divided into four subsamples, 310, 320, 330, and 340, respectively. In this embodiment, each subsample comprises from about 1000 to about 100,000 copies of the nucleic acid.

Partial Labeling of Polymers

Partial labeling of the polymer can be accomplished using chemical or enzymatic modifications. As shown in FIG. 1, polymer molecules 102 may be prepared by any technique known to one of ordinary skill in the art. In certain embodiments of the disclosed methods and devices, the polymer molecules 102 are naturally occurring DNA or RNA molecules, such as chromosomal DNA or messenger RNA (mRNA). Virtually any naturally occurring nucleic acid may be prepared and sequenced by the disclosed methods including, without limit, chromosomal, mitochondrial or chloroplast DNA or ribosomal, transfer, heterogeneous nuclear or messenger RNA. Nucleic acids to be sequenced may be obtained from either prokaryotic or eukaryotic sources by standard methods known in the art. Methods for preparing and isolating various forms of nucleic acids are known. (See e.g., Berger and Kimmel eds., Guide to Molecular Cloning Techniques, Academic Press, New York, N.Y., 1987; Sambrook, Fritsch and Maniatis, eds., Molecular Cloning: A Laboratory Manual, 2nd Ed., Cold Spring Harbor Press, Cold Spring Harbor, NY, 1989). Any method for preparation of template nucleic acids 200 known in the art may be used in the disclosed methods.

When polynucleic acids are used as the polymers, standard molecular biology techniques may be used to accomplish the partial labeling. Such methods are described in Sambrook et al. in Molecular Cloning: A Laboratory Manual. Cold Spring Harbor Laboratory Press, N.Y., Vol. 1-3 (1989) and D. Glover, DNA Cloning Volume I: A Practical Approach, IRL Press, Oxford, 1985. These techniques include, but are not limited to, a) random primer methods, b) polymerase chain reaction (PCR) methods, c) strand replacement methods, and d) primer extension methods.

The random primer method is based on the work of Feinberg (Anal. Biochem. 132: 6-13 (1983) Id. and 137: 266-267 (1984)). It is known that oligonucleotides can serve as primers for the initiation of DNA synthesis on single-stranded templates by DNA polymerases. If the oligonucleotides are heterogeneous in sequence, they will form hybrids at many positions, so that every nucleotide of the template except those at the extreme 5′ terminus will be copied at equal frequency into the product. By using labeled deoxynucleotide triphosphates (dNTPs) as precursors, labeled DNA molecules can be synthesized. Random primers can be obtained by: a) digesting calf thymus or salmon sperm DNA with DNAase I to generate a large population of single-stranded DNA fragments 6-12 nucleotides in length; b) purchasing random oligonucleotides from commercial sources (e.g. Pharmacia, Roche, International Biotehnologies etc.); or c) synthesizing on an automated DNA synthesizer a population of octamers or 9-mers that contains all four nucleotides in every position. Because of their uniform length and lack of sequence bias, synthetic oligonucleotides are preferred. The use of longer 9-mer primers and exonuclease-free enzyme results in higher labeling efficiency and longer probes. This method overcomes many of the disadvantages of conventional nick translation procedures while producing probes from small amounts of DNA (from about 10 to about 20 ng). Random Primer DNA labeling kits are commercially available from Panvera and other companies.

The type of DNA polymerase used depends on the nature of the template: a) RNA-dependent DNA polymerase (reverse transcriptase) is used to copy single-stranded RNA templates into cDNA or; b) the Klenow fragment of E. coli DNA polymerase I is used when the template is single stranded DNA. In both cases, the synthesis of DNA is carried out using one labeled type of dNTP and three unlabeled types of dNTPs as precursors to yield DNA wherein a large proportion of a particular type of nucleotide is labeled. Reverse transcriptase kits are commercially available from Qiagen GmbH (Germany) and other companies.

All of these techniques can be performed in one or two steps, depending on the polymerase used. For Klenow and reverse transcriptases, the labeling and primer extension/chain termination reactions can be combined by lowering the concentration of one of the four dNTPs and adding the same labeled dNTP. For all polymerases, including the widely-used T7 DNA polymerase, these two reactions can be performed sequentially. In the labeling reaction, the primer is extended a short time using limiting concentrations of dNTPs and a single labeled dNTP. In the extension/termination step, the extended primers are further extended in the presence of both dNTPs and ddNTPs, leading to sequence specific chain terminations. The principal advantage of this method is that multiple labels are incorporated into each chain and the density of the labels can be controlled by varying the ratios of labeled dNTPs with unlabeled dNTPs. In certain embodiments, from about 1% to about 50% of each type of nucleotide are labeled.

The PCR method for amplifying DNA is covered by U.S. Pat. Nos. 4,683195 and 4,683,202 assigned to Hoffman-La Roche Inc. and F. Hoffann-La Roche Ltd. In the PCR method, the resulting product can be labeled with either modified nucleotides or modified oligonucleotide primers. Typically, these labels are fluorescent labels because they offer the advantages of direct detection, sensitivity, and mulitcolor capability. Fluorescently labeled deoxynucleotide triphosphates (dNTPs) and fluorescently end-labeled oligonucleotide primers are commercially available for use in PCR product labeling from Molecular Dynamics. PCR primers labeled fluorescently at the 5′ end can be produced de novo during oligonucleotide synthesis or by using chemistries such as the Fluorescent 5′-Oligolabeling Kit from Amersham Pharmacia Biotech. In contrast to incorporation of multiple labeled dNTPs, PCR with labeled primers results in a fixed number of labels (one or two) per DNA. While this provides less sensitivity than labeling with fluorescent dNTP, amplification with labeled primers does offer a quantitative advantage because the molar ratio of label to final product is known.

In the strand replacement method, a DNA polymerase catalyzes the exchange (replacment) of an unlabeled nucleotide with a labeled nucleotide. In the presence of only one dNTP the 3′→5′ exonuclease function will degrade a strand of double-stranded DNA from the 3′ hydroxyl terminus until a nucleotide is exposed that is complementary to the dNTP present. A continuous series of synthesis and exchange reations will then take place. For example, if 10% of a dNTP (such as dATP) is fluorescein-labeled, the resulting DNA will have fluoroscein-labeled nucleotide (A) in approximately every 40th position because there are four types of nucleotides in a DNA molecule. But if there is only one type of nucleotide, then the labeling will occur every 10^{th} position for 10% labeling. Bacteriophage T4 DNA polymerase is commercially available from many vendors. The most popular ones are New England Biolabs (Beverly, Mass.) and Worthington Biochem (Lakewood N.J.). Any particular type of label for example, radioactive labels, fluorescent labels, and the like, can be used in any of these DNA labeling methods and subsequently used in the sequencing method of the disclosed methods and devices.

In the “primer extension” method, the primer has a specific sequence and will initiate polymerization to a desired location of the target sequence (the “template”). This method is more preferred than the random primer method. Random primer is a good labeling method for making probe in molecular hybridization application, but not as a good method of generating products for sequencing because it will create many short fragments from a large template.

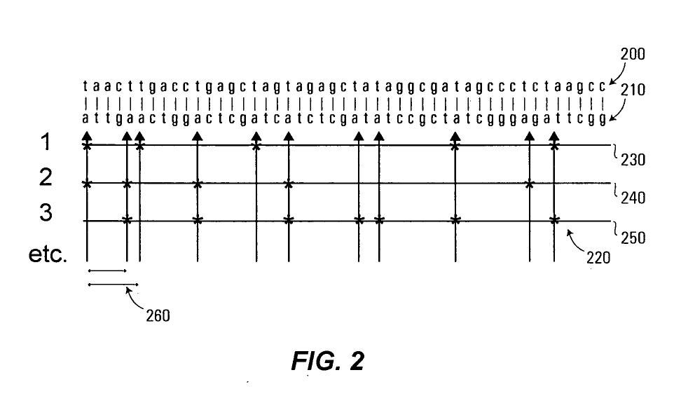

As shown in FIG. 2, certain embodiments of the disclosed methods and devices concern synthesis of a partially labeled complementary strand 230, 240, 250 of DNA to be sequenced. The template strand 200 can be either RNA or DNA. With an RNA template strand 200, the synthetic reagent may be a reverse transcriptase, examples of which are known in the art. In embodiments where the template strand 200 is a molecule of DNA, the synthetic reagent may be a DNA polymerase, examples of which are known in the art. In other embodiments of the disclosed methods and devices, the complementary strand 230, 240, 250 can be a molecule of RNA. This requires that the synthetic reagent be an RNA polymerase. In these embodiments, no primer is required. However, the template strand 200 should contain a promoter that is effective to bind RNA polymerase and initiate transcription of an RNA complementary strand 230, 240, 250. Optimization of promoters is known in the art. The embodiments of the disclosed methods and devices are not limited as to the type of template molecule 200 used, the type of complementary strand 230, 240, 250 synthesized, or the type of polymerase utilized. Virtually any template 200 and any polymerase that can support synthesis of a nucleic acid molecule complementary 230, 240, 250 in sequence 210 to the template strand 200 may be used.

In some embodiments of the disclosed methods and devices, functional groups, such as labels, may be covalently attached to cross-linking agents so that interactions between template strand 200, complementary strand 230, 240, 250 and polymerase may occur without steric hindrance. Alternatively, the nucleic acids may be attached to surfaces using cross-linking agents. Typical cross-linking groups include ethylene glycol oligomers and diamines. Attachment may be by either covalent or non-covalent binding. Various methods of attaching nucleic acid molecules to surfaces are known in the art and may be employed.

Each subsample may contain a labeled nucleotide precursor in order to produce randomly labeled complementary strands 230, 240, 250. Nucleotide precursors covalently attached to a variety of labels, such as fluorescent labels, may be obtained from standard commercial sources (e.g., Molecular Probes, Inc., Eugene, Oreg.). Alternatively, labeled nucleotide precursors may be prepared by standard techniques well known in the art. Any known method for preparing labeled nucleotide precursors may be used in the practice of the claimed subject matter.

In a non-limiting example, the percentage of labeled nucleotide precursors added to a particular reaction is 10%, although it is contemplated that the percentage of labeled nucleotide precursors in a reaction range from about 0.5 to about 85% of the total amount of the same type of nucleotide in that reaction. For example, where the reaction contains a labeled adenosine nucleotide precursor, the reaction may contain 10% labeled adenosine nucleotide and 90% unlabeled adenosine nucleotide, along with unlabeled cytosine, guanine and thymidine nucleotides.

The use of a lower percentage of labeled nucleotide 220 results in “signal stretching.” Signal stretching decreases the density of detectable signals as compared to a completely labeled monomer-type in a polymer. The normal distance between two adjacent nucleotides is ⅓ nm. If 10% of nucleotide precursors are labeled, then the average distance between two adjacent labeled nucleotides 220 in the complementary nucleic acid 230, 240, 250 will be approximately 13.6 nm. Stretching out the distance between adjacent labeled nucleotides 220 allows detection by techniques such as conductivity measurement, spectrophotometric analysis, AFM or STM. Such methods cannot distinguish between labels that are ⅓ nm apart. A label may be detected using any detector known in the art, such as a spectrophotometer, luminometer, NMR (nuclear magnetic resonance), mass-spectroscopy, imaging systems, charge coupled device (CCD), CCD camera, photomultiplier tubes, avalanche photodiodes, AFM or STM.

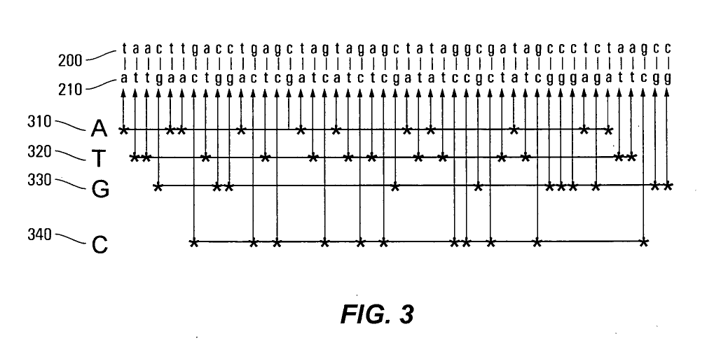

While the distance between the labels 220 is inevitably used, the disclosed methods actually measure time between labels 220 or the frequency of detection. The relationship between time and distance between labels 220 is proportional or linear. Thus, the time maps of FIGS. 2 and 3 are used to determine the distances between the labels 220.

In one embodiment, each strand is labeled such that the average time between two adjacent labeled monomers is significantly longer than the average time between two adjacent prelabeled monomers of the same type in the polymer subsample.

In various embodiments of the disclosed methods and devices, a nucleotide precursor with an incorporated reactive group and/or hapten may be attached to a secondary label, such as an antibody. Any type of detectable label known in the art may be used, such as Raman tags, fluorophores, chromophores, radioisotopes, enzymatic tags, antibodies, chemiluminescent, electroluminescent, affinity labels, etc. One of skill in the art will recognize that these and other known label moieties not mentioned herein can be used in the disclosed methods.

The label moiety to be used may be a fluorophore, such as Alexa 350, Alexa 430, AMCA (7-amino-4-methylcoumarin-3-acetic acid), BODIPY (5,7-dimethyl-4-bora-3a, 4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine-2), Cy3, Cy5, 5-carboxyfluorescein, fluorescein, 6-JOE (2′7′-dimethoxy-4′5′-dichloro-6-carboxyfluorescein), Oregon Green 488, Oregon Green 500, Oregon Green 5, Pacific Blue, Rhodamine Green, Rhodamine Red, ROX (6-carboxy-X-rhodamine), TAMRA (N,N,N′,N′-tetramethyl-6-carboxyrhodamine), tetramethylrhodamine, and Texas Red. Fluorescent or luminescent labels can be obtained from standard commercial sources, such as Molecular Probes (Eugene, Oreg.).

In certain embodiments of the disclosed methods and devices, nucleotides may be labeled with a bulky group. Non-limiting examples of such bulky groups include antibodies, quantum dots and metal groups. The antibodies may be labeled with a detectable marker. The detectable marker may be selected from the group consisting of enzymes, paramagnetic materials, avidin, streptavidin or biotin, fluorophores, chromophores, chemiluminophores, heavy metals, and radioisotopes.

In some embodiments, nanoparticles may generate unique optical signals such as surface plasmon resonances or surface-enhanced Raman scattering signals. One example of such nanoparticles are complex organic-inorganic nanoparticles (COINs) that are currently under development.

Metal groups used as labels may consist of one type of metal, such as gold or silver, or a mixture of metals. In particular embodiments of the disclosed methods and devices, metal groups may comprise nanoparticles. Methods of preparing nanoparticles are known (e.g., U.S. Pat. Nos. 6,054,495; 6,127,120; 6,149,868; Lee and Meisel, J. Phys. Chem. 86:3391-3395, 1982). Nanoparticles may also be obtained from commercial sources (e.g., Nanoprobes Inc., Yaphank, N.Y. and Polysciences, Inc., Warrington, Pa.). In some embodiments, nanoparticles may be cross-linked to each other prior to attachment to nucleotides. Methods of cross-linking of nanoparticles are known in the art. (e.g. Feldheim, “Assembly of metal nanoparticle arrays using molecular bridges,” Electrochemical Society Interface, Fall, 2001, pp. 22-25.) Cross-linked nanoparticles comprising monomers, dimers, trimers, tetramers, etc. may be used, for example, to provide distinguishable mass labels for different types of nucleotides. Although nanoparticles of any size are contemplated, in specific embodiments of the disclosed methods and devices the nanoparticles may be about 0.5 to 5 nm in diameter.

Antibodies used as bulky groups may also be labeled with nanoparticles. Gold nanoparticles are available with a maleimide functionality on the surface which allows covalent linkage to antibodies, proteins and peptides through sulfhydryl groups. (Monomaleimido NANOGOLD®, Integrated DNA Technologies, Coralville, Iowa.) Techniques to label antibodies and/or nucleotides with nanoparticles are known. For example, antibodies may be labeled with gold nanoparticles after reducing the disulfide bonds in the hinge region with a mild reducing agent, such as mercaptoethylamine hydrochloride (MEA). After separation of the reduced antibody from MEA, it can be reacted with Monomaleimido NANOGOLD®. Gel exclusion chromatography can be utilized to separate the conjugated antibody from the free gold nanoparticles. Antibody fragments can be labeled in a similar manner. Gold nanoparticles can also be attached directly to labeled nucleotide precursors that contain a thiol group.

Primers may be obtained by any method known in the art. Generally, primers are between ten and twenty bases in length, although longer primers may be employed. In certain embodiments of the disclosed methods and devices, primers are designed to be exactly complementary to a known portion of a template nucleic acid 200. In one embodiment of the disclosed methods and devices, primers are located close to the 3′ end of the template nucleic acid 200. Methods for synthesis of primers of any sequence, for example using an automated nucleic acid synthesizer employing phosphoramidite chemistry are known and such instruments may be obtained from standard sources, such as Applied Biosystems (Foster City, Calif.) or Millipore Corp. (Bedford, Mass.).

Other embodiments of the disclosed methods and devices, involve sequencing a nucleic acid in the absence of a known primer-binding site. In such cases, it may be possible to use random primers, such as random hexamers or random oligomers of 7, 8, 9, 10, 11, 12, 13, 14, 15 bases or greater length, to initiate polymerization of a complementary strand 230, 240, 250. To avoid having multiple polymerization sites on a single template strand 200, primers besides those hybridized to the template molecule 200 near its attachment site to an immobilization surface may be removed by known methods before initiating the synthetic reaction.

As mentioned previously, it is very difficult to label two monomers directly adjacent to each other, or to directly detect two labeled monomers directly adjacent to each other. This method avoids these problems by not requiring labeling of every monomer type in a particular polymer molecule.

Sequencing Device

Returning to FIG. 1, a sequencing device 100 may be used to perform the sequencing analysis. A sequencing device contains a single strand of a partially labeled polymer. In some embodiments of the disclosed methods and devices, the partially labeled polymer 102 may be attached to an immobilization surface 109 within the sequencing device 100 before cleavage into individual monomers 110.

Techniques to immobilize the partially labeled nucleic acid molecule 102 on surfaces 109 are well known in the art. A surface, such as functionalized glass, including but not limited to silanized, gold-coated, avidin- or streptavidin-coated, or otherwise derivatized glass, silicon, PDMS (polydimethlyl siloxane), gold, silver or other metal coated surfaces, quartz, plastic, PTFE (polytetrafluoroethylene), PVP (polyvinyl pyrrolidone), polystyrene, polypropylene, polyacrylamide, latex, nylon, nitrocellulose, or any other material known in the art that is capable of attaching to nucleic acids, may be immersed in a reaction chamber and a modified end, such as thiol modified or biotin modified, of the labeled nucleic acids 102 may be allowed to bind to the surface. In some embodiments of the disclosed methods and devices, the nucleic acid molecules can be oriented on a surface as disclosed below.

In certain embodiments, the sequencing device 100 will be designed to hold the polymer in solution and/or be temperature controlled, for example by incorporation of Pelletier elements or other methods known in the art. In embodiments that relate to nucleic acid sequencing, methods of controlling temperature for low volume liquids are known in the art (e.g., U.S. Pat. Nos. 5,038,853, 5,919,622, 6,054,263 and 6,180,372).

In certain embodiments, the sequencing device comprises one or more fluid channels, for example, to provide connections to a molecule dispenser, to a waste port, to a polymer loading port, and/or to the source for cleaving off individual monomers. All these components may be manufactured in a batch fabrication process, as known in the fields of computer chip manufacture or microcapillary chip manufacture. In some embodiments of the disclosed methods and devices, the apparatus 100 and its individual components may be manufactured as a single integrated chip 101. Such a chip may be manufactured by methods known in the art, such as by photolithography and etching. However, the manufacturing method is not limiting and other methods known in the art may be used, such as laser ablation, injection molding, casting, or imprinting techniques. Methods for manufacture of nanoelectromechanical systems may be used for certain embodiments of the disclosed methods and devices. (See e.g., Craighead, Science 290:32-36, 2000.) Microfabricated chips are commercially available from sources such as Caliper Technologies Inc. (Mountain View, Calif.) and ACLARA BioSciences Inc. (Mountain View, Calif.).

The material comprising the apparatus 100 and its components may be selected to be transparent to electromagnetic radiation at excitation and emission frequencies used for the detection unit 107. Glass, silicon, and any other materials that are generally transparent in the visible frequency range may be used for construction of the apparatus 100.

In other embodiments of the disclosed methods and devices, portions of the apparatus 100 and/or accessory devices may be designed allow access of the detection unit to measure the times between labeled groups.

Cleavage of Monomers from the Polymer Subsample.

Still referring to FIG. 1, the polymer will be sequentially cleaved into individual labeled and unlabeled monomers by chemical or enzymatic means. This is typically accomplished by first immobilizing the polymers 102 on a solid support 109 in a system equipped for sequencing and detection 100. Then, using a combination of chemical or enzymatic methods and microfluidics, each monomer 110 (both labelled and non-labelled) from the polymer strand is sequentially cleaved and transported into a collection volume for detection. For example, if the polymer molecule is a nucleic acid, randomly, partially labeled nucleotides of a DNA strand may be attached to the outer surface of polystyrene bead or some other type of molecular carrier 109. The bead is captured or held in place in a microfluidic channel of the system, using optical tweezers, a restriction channel, or a some other mechanical attachment such that a single molecule 102 is positioned in the reaction chamber of the sequencing device. For example, nucleic acid molecules can be cleaved with an exonuclease in an aqueous environment of the device.

Examples of suitable exonucleases, include, but are not limited to exonuclease 1, lambda exonuclease, or a DNA polymerase with exonuclease activity, such as T4 DNA polymerase or T7 DNA polymerase. Exonuclease I digests single stranded DNA from the 3′ to 5′ end; lambda exonuclease digests double stranded DNA from the 5′ to 3′ end; and T4 DNA polymerase (exonuclease)and T7 DNA polymerase (exonuclease)digest single and double stranded DNA from the 3′ to 5′ end.

A buffered enzyme solution with exonuclease activity 103 is then flowed using a flow control device into the reaction chamber of the channel to digest the DNA strand and release the individual labeled or unlabeled nucleotide monomers 110 one at a time. Preferably this enzyme solution is pumped into the reaction chamber at a predetermined rate using the flow control device. The cleaved nucleotide monomers are carried/transported in the flow a directed through a sample cell 90 where the signal from the label is sequentially detected as a function of time T. The nucleotide monomers are evenually carried/transported to a collection or waste chamber 80.

The rate of digestion may be impeded when it encounters a labeled base, the degree of which depends on the structure of the label and the particular cleavage method used (e.g. enzymatic, chemical, and the like).

Detection

The labels of sequentially separated labeled monomers from each polymer subsample are then detected using single molecule detection techniques as a function of time since the initiation of cleavage. The monomers can be detected by a variety of techniques and the embodiments of the disclosed methods and devices are not limited by the type of detection unit used; any known detection unit may be used in the disclosed methods and apparatus 100. For example in certain embodiments of the disclosed methods and devices a detection unit 107 may comprise an optical device, such as a scanning probe microscope (SPM) for example a magnetic force microscope, lateral force microscope, force modulation microscope, phase detection microscope, electrostatic force microscope, scanning thermal microscope, or a near-field scanning optical microscope, or the like. In certain embodiments an atomic force microscope (AFM), a scanning tunneling microscope (STM), an electrical detector, a spectrophotometric detector, or the like may be used. Methods of use of such detection units are well known in the art.

In certain embodiments, nanopore detection technology may be used. Nanopores measure the changes in ionic conductivity when a particular type of molecule passes through a it or membrane channel containing nanopores. Nanopore diameters are typically on the order of a few nanometers. The nanopore is filled only in an electrolyte solution and a voltage bias induced by a cathode and anode arrangement causes ions to flow through the nanopore in the sample cell 90. The ionic current flow is on the order of picoamperes. When single molecules are drawn into the nanopore by the voltage bias, the molecules partially obstruct the nanopore and reduce its ionic conductivity. Quantifying the reduction of the ionic conductivity allows for the direct characterization of a labeled or unlabeled monomer on a nanosecond or microsecond time scale without the need for amplification. The sensitivity of this technique can be increased by covalently tethering a molecule near the pores lumen to act as an additional sensor that can selectively, but reversibly, bind to the different types of molecules to be analyzed. For example, when a molecule that more strongly interacts with the sensor molecule is drawn into the lumen of a nanopore by the voltage bias, it is more likely to have an interaction with the sensor molecule that increases its time in the nanopore and creates a signature time duration of ionic conductivity reduction. Likewise, when a molecule that only weakly interacts with the sensor molecule is drawn into the lumen of a nanopore, its time in the nanopore is not signficantly increased, again creating a signature time duration of ionic conductivity reduction. Plotting the translocation duration vs. the change in ionic conductivity allows for the identification of each unique type of labeled or unlabeled monomer. Examples of such sensor molecules for nucleotide monomers include a binding molecule for the label or a base pair complement to the nucleotide. Nanopores have been used to sequence codons in a single molecule of DNA (See Wang et al. Nature Biotechnology, 19: 622-623 (2001); Meller et al. Proc. Nat'l. Acad. Sci. 97: 1079 (2000)). A labeled nucleotide can have a larger size and different chemical properties compared to normal nucleotides.

In alternative embodiments, labeled nucleotides 220 attached to luminescent labels may be detected using a light source and photodetector, such as a diode-laser illuminator and fiber-optic or phototransistor detector (see Sepaniak et al., J. Microcol. Separations 1:155-157, 1981; Foret et al., Electrophoresis 7:430-432, 1986; Horokawa et al., J. Chromatog. 463:39-49 1989; U.S. Pat. No. 5,302,272.) Other exemplary light sources include vertical cavity surface-emitting lasers, edge-emitting lasers, surface emitting lasers and quantum cavity lasers, for example a Continuum Corporation Nd-YAG pumped Ti:Sapphire tunable solid-state laser and a Lambda Physik excimer pumped dye laser. Other exemplary photodetectors include photodiodes, avalanche photodiodes, photomultiplier tubes, multianode photomultiplier tubes, phototransistors, vacuum photodiodes, silicon photodiodes, and charge-coupled devices (CCDs). Using surface-enhanced Raman scattering, fluorescence and other optical methods, single nucleotide molecules can be detected and identified (see Kneipp et al., Phys. Rev. E, 57: R6281 (1998); Keir et al., Anal. Chem., 74: 1503 (2002); Doering et al., J. Phys. Chem. B, 106: 311 (2002)).

In some embodiments, the photodetector, light source, and nanopore may be fabricated into a semiconductor chip using known N-well Complementary Metal Oxide Semiconductor (CMOS) processes (Orbit Semiconductor, Sunnyvale, Calif.). In alternative embodiments of the disclosed methods and devices, the detector, light source and nanopore may be fabricated in a silicon-on-insulator CMOS process (e.g., U.S. Pat. No. 6,117,643). In other embodiments of the disclosed methods and devices, an array of diode-laser illuminators and CCD detectors may be placed on a semiconductor chip (U.S. Pat. Nos. 4,874,492 and 5,061,067; Eggers et al., BioTechniques, 17: 516-524, 1994).

In certain embodiments, a highly sensitive cooled CCD detector may be used. The cooled CCD detector has a probability of single-photon detection of up to 80%, a high spatial resolution pixel size (5 microns), and sensitivity in the visible through near infrared spectra. (Sheppard, Confocal Microscopy: Basic Principles and System Performance in: Multidimensional Microscopy, Springer-Verlag, New York, N.Y., pp. 1-51, 1994.) In another embodiment of the disclosed methods and devices, a coiled image-intensified coupling device (ICCD) may be used as a photodetector that approaches single-photon counting levels (U.S. Pat. No. 6,147,198). A small number of photons triggers an avalanche of electrons that impinge on a phosphor screen, producing an illuminated image. This phosphor image is sensed by a CCD chip region attached to an amplifier through a fiber optic coupler. In some embodiments of the disclosed methods and devices, a CCD detector on a chip may be sensitive to ultraviolet, visible, and/or infrared spectra light (e.g., U.S. Pat. No. 5,846,708).

In some embodiments, a nanopore may be operably coupled to a light source and a detector on a semiconductor chip. In certain embodiments of the disclosed methods and devices, the detector may be positioned perpendicular to the light source to minimize background light. The photons generated by excitation of a luminescent label may be collected by a fiber optic. The collected photons are transferred to a CCD detector and the light detected and quantified. The times at which labeled nucleotides 220 are detected may be recorded and nucleotide time maps 310, 320, 330, 340 may be constructed. Methods of placement of optical fibers on a semiconductor chip in operable contact with a CCD detector are known (e.g., U.S. Pat. No. 6,274,320).

In some embodiments, an avalanche photodiode (APD) may be made to detect low light levels. The APD process uses photodiode arrays for electron multiplication effects (e.g., U.S. Pat. No. 6,197,503). In other embodiments of the disclosed methods and devices, light sources, such as light-emitting diodes (LEDs) and/or semiconductor lasers may be incorporated into semiconductor chips (e.g., U.S. Pat. No. 6,197,503). Diffractive optical elements that shape a laser or diode light beam may also be integrated into a chip.

In certain embodiments of the disclosed methods and devices, a light source produces electromagnetic radiation that excites a photo-sensitive label, such as fluorescein, attached to a nucleic acid. In some embodiments of the disclosed methods and devices, an air-cooled argon laser at 488 nm excites fluorescein-labeled nucleic acid molecules 230, 240, 250. Emitted light may be collected by a collection optics system comprising an optical fiber, a lens, an imaging spectrometer, and a thermoelectrically-cooled CCD camera or a liquid nitrogen cooled CCD camera. Alternative examples of fluorescence detectors are known in the art.

Assembling a Complete Monomer-Time Map for each Polymer Sub-Sample

One method for assembling monomer-time maps comprises: 1) obtaining a cleaving time for each monomer (labeled and unlabeled); 2) collecting multiple monomer-time maps over multiple runs for each type of monomer; 3) adjusting for the difference in time required to cleave each labeled monomer and unlabeled monomer; 4) comparing multiple runs and marking when each monomer is detected; 5) blocking the time segments required to cleave each monomer; 6) repeating steps 2 to 4 for all types of monomers; and 7) assembling the monomer-time maps for all types of monomers to produce monomer-position map.

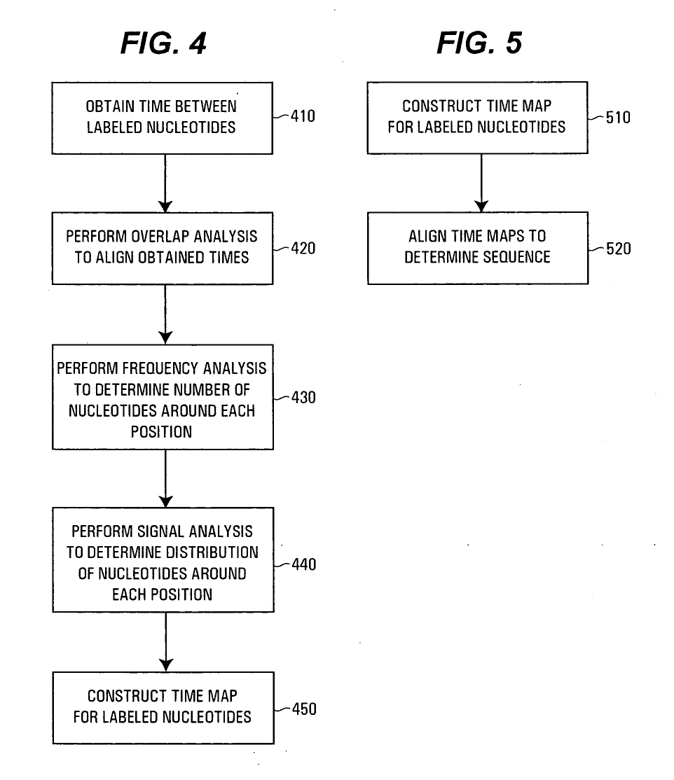

For example, as shown in FIGS. 2-4, the time between each labeled nucleotide 220 data for each strand cleaved in the reaction chamber 110 may be collected and analyzed (FIG. 2). A time map 310, 320, 330, 340 may be constructed 450 for each strand in the subsample of each monomer type 110 and the resulting time maps 310, 320, 330, 340 may be aligned 520 to produce a nucleic acid sequence 210 (FIG. 3). The time maps 310, 320, 330, 340 for each monomer type 110, 120, 130, 140 may be constructed at 450 as shown in FIG. 4 by overlapping 420, frequency 430 and signal 440 analysis of the time between labeled nucleotides 220. The time maps 310, 320, 330, 340 may be aligned 520 into a sequence 210 by non-overlapping data analysis. Time maps 310, 320, 330, 340 may be constructed at 450 and aligned 520 by an information processing and control system, for example, a computer.

Determination of the Polymer Sequence: Nucleic Acids

In some embodiments, determining the sequence 210 of a polymer 230, 240, 250 includes constructing time maps 310, 320, 330, 340 for each type of labeled monomer 220 and aligning 520 the time maps 310, 320, 330, 340 to produce the complete sequence 210. Referring to FIG. 2, the data from each partially labeled nucleotide subsample run can be assembled into a complete nucleotide-time measurement map for each nucleotide type. Computerized methods can be used to reconstruct the monomer-time separation map for each monomer type (e.g. in DNA sequencing, the nucleotides A, G, C, and T).

Referring to FIG. 3, the data from each of the monomer-time maps can then be integrated into a complete polymer sequence. This is also conveniently done using computerized algorithms/methods. By aligning these maps for non-overlapping regions, the complete DNA sequence can be determined.

The sequence of the template nucleic acid 200 will be exactly complementary to the determined sequence 210.

In an exemplary embodiment, a time map 310, 320, 330, 340 for each type of labeled nucleotide 220 is constructed 450 according to the process of FIG. 4. Random labeling and detection of labeled nucleotides 220 is performed on the complementary strands 230, 240, 250, resulting in a percentage of the nucleotides in each strand 230, 240, 250 being labeled 220. The percentage of labeled nucleotides 220 may vary depending on the labeling and detection schemes used. In one embodiment, about 10% of the nucleotides of the same type on a complementary strand 230, 240, 250 are labeled 220, in order to ensure easily detectable times between labeled nucleotides 220. After synthesis of labeled complementary strands 230, 240, 250, the time between labeled nucleotides 220 are obtained 410 (FIG. 2). The obtained times 410 between labeled nucleotides 220 are represented graphically in FIG. 2. The obtained times 410 may be used to construct 450 time maps 310, 320, 330, 340 for each type of labeled nucleotide 220.

In various embodiments of the disclosed methods and devices, overlap analysis is performed at 420 on the obtained times. This serves to align the times so that the complementary strands 230, 240, 250 begin and end at the same place.

In one embodiment, the overlap analysis 420 comprises maximizing positional overlaps. Because a large number of strands 230, 240, 250 are used, each nucleotide in the sequence 210 will be labeled a large number of times in different strands 230, 240, 250. The basic idea of this “maximum overlap data analysis,” is to align the time-maps of the multiple runs from the same subsample to construct the shortest overlapping map. In this approach, positions with a high degree of overlap or “hotspots” are identified by comparing a particular position with a theoretical count for that position. The theoretical count can be calculated statistically based on the percentage of nucleotides that are labeled in a strand and the number of runs. Identification of a hotspot indicates the likely presence of a nucleotide in that position. By maximizing the alignment of hotspot times 420, the positions of labeled nucleotides 220 on each strand 230, 240, 250 will correspond.

In other embodiments, other methods for aligning the sequences are used. For example, each strand can be uniquely labeled at the beginning or the end of the strands 230, 240, 250 to align the obtained times at 420 in FIG. 4. This method may be used instead of or in addition to overlap analysis to align the obtained times 420. After the obtained times are aligned at 420, frequency analysis may be performed at 430 to determine the number of labeled nucleotides 220 around each labeled position. In one embodiment, using the law of large numbers and the independent and uniform nature of the labeling and detection processes, it can be inferred that a labeled nucleotide 220 in each position is labeled with approximately the same probability on each complementary strand 230, 240, 250 as the probability of being labeled on a single strand. Thus, in the example using 10% labeled nucleotides 220, if 1000 strands 230, 240, 250 are used, the number of times a nucleotide at a single position is labeled 220 should be 10% of 1000, or 100 times. This analysis can be done by a computer program as is known by those skilled in the art.

Using this observation, the number of labeled nucleotides 220 that are too close for independent detection can be determined. For example, using 10% labeling probability and 1000 labeled complementary strands 230, 240, 250, if 102 strands 230, 240, 250 show a labeled nucleotide 220 at a given position, then it can be inferred that that position is occupied by one labeled nucleotide 220, with no other labeled nucleotides 220 so close that detection errors occur. On the other hand, if 197 strands 230, 240, 250 show a labeled nucleotide 220 at the given position, it can be inferred that there are two labeled nucleotides 220 present, one in the given position and a second too close, to accurately measure. The labeled nucleotides 220 of the same type may be contiguous with each other or may be spaced apart by one or more nucleotides of a different type. The same analysis applies where two or more nucleotides of the same type are located in adjoining positions. Where two adjacent nucleotides are identical, the position would be expected to be labeled about twice as often, three adjacent identical nucleotides should be labeled about three times as often, etc.

When it is determined that two or more labeled nucleotides 220 are located too close together for independent measurement, signal analysis may be performed at 440 to determine the spatial relationship. For example, the signal produced by two labeled nucleotides 220 separated by one other nucleotide may be different from the signal produced by two contiguous labeled nucleotides 220, or two labeled nucleotides 220 separated by two other nucleotides.

Other methods may also be used to distinguish the spatial relationship between closely spaced identical nucleotides. In certain embodiments, frequency analysis may be performed 430 to determine the relative positions of labeled nucleotides 220. For example, to distinguish between three labeled nucleotides 220 in a row and two labeled nucleotides 220 separated by one other nucleotide, one signal should occur only two-thirds as often as the other signal. Frequency and signal analysis may be performed in any order in the claimed methods.

As disclosed in FIG. 4 and FIG. 5, a time map 310, 320, 330, 340 (FIG. 3) for each type of labeled nucleotide 220 may be constructed att 450. Although all four types of nucleotides may be labeled 220 and time maps 310, 320, 330, 340 constructed 450, in alternative embodiments of the disclosed methods and devices only three of the four types of nucleotides may be labeled 220 and analyzed. In such embodiments, the positions of the fourth type of nucleotide may be inferred by gaps in the time maps 310, 320, 330, 340 aligned at 520 for the other three types of nucleotide. The complete nucleic acid sequence 210 may be determined by the aligning step at 520 of the time maps 310, 320, 330, 340 for the different types of labeled nucleotides 220.

In certain embodiments of the disclosed methods and devices, the time maps 310, 320, 330, 340 may be aligned at 520 using the non-overlapping rule and minimum non-overlap data analysis. According to the non-overlapping rule, two different nucleotides cannot occupy the same position in the sequence 210. According to “minimum non-overlap” data analysis, the shortest sequence that contains no overlapped point is is used to align the time-maps of from each of the four nucleotide subsamples. This can be easily done by a computer program and would be apparent to those skilled in the art. The presence of any overlapped point indicates the presence of problematic sites that need to be further sequenced (i.e. repeating the experiments).

In some embodiments of the disclosed methods and devices, time maps 310, 320, 330, 340 may be aligned at 520 one at a time, beginning with the time map 310, 320, 330, 340 with the greatest number of labeled nucleotides 220. If more than one possible alignment is found, the alignment producing the shortest sequence 210 is chosen, according to the rule of minimum sequence 210 length. If additional time maps 310, 320, 330, 340 cannot be aligned 520 without overlap, the alignments may be iteratively reevaluated until an alignment without overlap is obtained. If no alignment of the time maps 310, 320, 330, 340 exists such that the non-overlap rule is completely observed, then alternative constructions are generated at 450 for the time maps 310, 320, 330, 340 that may also be iteratively reevaluated until a non-overlapping sequence 210 is obtained.

In certain embodiments of the disclosed methods and devices, the sequencing process may produce a perfectly aligned sequence 210 for most of the nucleic acid, with one or more short segments where overlap occurs or where the sequence 210 is otherwise ambiguous. The operator may review the data at any point in the analysis and conclude that either the entire nucleic acid should be sequenced again, or that only short regions of the nucleic acid template 200 should be resequenced. Such evaluation of the results of sequence 210 analysis is well within the ordinary skill in the art, as is known with existing methods of nucleic acid sequencing. This determination may be made automatically by a computer based on statistical analysis of the data, or by a human user.

In another embodiment of the disclosed methods and devices, the beginning and ends of the time maps 310, 320, 330, 340 as they relate to the sequence 210 may be known. In this case, the alignment at 520 may include lining up the known ends of the time maps 310, 320, 330, 340.

The minimum number of runs per subsample is equivalent to the level of labeling redundancy divided by the percentage of labeling. For example, with 10-fold redundancy in a 10% labeling reaction, the number of run is 10/0.1=100 (per subsample).

Alternatively, the minimum number of runs per subsample can be calculated from the acceptable rate of error and the labeling efficiency. The labeling efficiency p is a number between 0 and 1; wherein when p is 1, labeling is 100% and when p is 0, the labeling is 0%. The probability that one specific position of a monomer is not labeled in n runs is (1−p)\hat{ }n. If the labeling efficiency is 10% (p=0.1), the chance that one monomer is not labeled within 100 runs is 0.000027 (=0.9\hat{ }100 , or 0.0027%. This implies that there will be, on average, one missing monomer for every 37,649 (=1/0.000027) monomers with 100 runs.

In this sequencing method, once partially labeled “A”, “G”, and “T” (or any combination of three nucleotides) are successfully detected, the last nucleotide (for example “C”) can serve as verification of the sequence.

If only two labeled nucleotides are detected (for example A and T), the complete sequence can be determined by doing multiple runs on both the DNA and cDNA strands and assembling the map with a computer. Due to the base pairing rule, where A pairs with T and G pairs with C, the entire oligo-sequence can be determined.

Information Processing and Control System and Data Analysis

In certain embodiments of the disclosed methods and devices, the sequencing apparatus 100 of FIG. 1 may comprise an information processing and control system 108 and 111. The embodiments are not limiting for the type of information processing and control system used. An exemplary information processing and control system may incorporate a computer 108 comprising a bus for communicating information and a processor for processing information. In one embodiment of the disclosed methods and devices, the processor is selected from the Pentium® family of processors, including without limitation the Pentium® II family, the Pentium® III family and the Pentium® 4 family of processors available from Intel Corp. (Santa Clara, Calif.). In alternative embodiments of the disclosed methods and devices, the processor may be a Celeron®, an Itanium®, a Pentium Xeon® or an X-scale processor (Intel Corp., Santa Clara, Calif.). In various other embodiments of the disclosed methods and devices, the processor may be based on Intel® architecture, such as Intel® IA-32 or Intel® IA-64 architecture. Alternatively, other processors may be used.

The computer 108 may further comprise a random access memory (RAM) or other dynamic storage device, a read only memory (ROM) and/or other static storage and a data storage device such as a magnetic disk or optical disc and its corresponding drive. The information processing and control system may also comprise other peripheral devices known in the art, such a display device (e.g., cathode ray tube or liquid crystal display), an alphanumeric input device (e.g., keyboard), a cursor control device (e.g., mouse, trackball, or cursor direction keys) and a communication device (e.g., modem, network interface card, or interface device used for coupling to an ethernet, token ring, or other types of networks).

In particular embodiments of the disclosed methods and devices, the detection unit 107 may also be coupled to the bus. Data from the detection unit may be processed by the processor and the data stored in the main memory. The processor may calculate times between labeled nucleotides 220, based on the time intervals between detection of labeled nucleotides 220. Nucleotide times may be stored in main memory and used by the processor to construct the time maps 310, 320, 330, 340 at 450 from each reaction. The processor may also align 520 the time maps 310, 320, 330, 340 at 520 to generate a nucleic acid sequence 210, from which a nucleic acid sequence 200 may be derived.

It is appreciated that a differently equipped information processing and control system than the example described herein may be used for certain implementations. Therefore, the configuration of the system may vary in different embodiments of the disclosed methods and devices. It should also be noted that, while the processes described herein may be performed under the control of a programmed processor, in alternative embodiments of the disclosed methods and devices, the processes may be fully or partially implemented by any programmable or hardcoded logic, such as field programmable gate arrays (FPGAs), TTL logic, or application specific integrated circuits (ASICs), for example. Additionally, the method may be performed by any combination of programmed general purpose computer components and/or custom hardware components.

In certain embodiments of the disclosed methods and devices, custom designed software packages may be used to analyze the data obtained from the detection unit 107. In alternative embodiments of the disclosed methods and devices, data analysis may be performed, using an information processing and control system and publicly available software packages. Non-limiting examples of available software for DNA sequence 210 analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information.

Advantages over prior methods of nucleic acid sequencing include the ability to read long nucleic acid sequences 210 in a single sequencing run, greater speed of obtaining sequence 210 data (up to 3,000,000 bases per second), decreased cost of sequencing and greater efficiency in terms of the amount of operator time required per unit of sequence 210 data generated.

### EXAMPLES

### DNA Sequencing

The following example is included to demonstrate particular embodiment of the disclosed methods and devices. However, those of skill in the art should, in light of the present disclosure, will appreciate that this is only one method and many changes can be made in the specific details which are disclosed and still obtain a like or similar result without departing from the claimed subject matter.

A 1.2 μg sample of genomic DNA is digested with a restriction enzyme and such that approximately 400,000 copies of a fragment of interest is isolated. The amount of genomic DNA to be digested can be increased to equate to 400,000 copies. The isolated fragment is divided into 4 sub-samples designated A, G, T and C. For a given DNA sub-sample, A, G, T, or C is partially labeled. Each subsample of partially labeled DNA 230 is immobilized on surface (see U.S. Pat. Nos. 5,840,862; 6,054,327; 6,225,055; 6,265,153; 6,303,296; 6,344,319) and a single DNA strand is isolated.

For a given target DNA, the labeled and unlabeled nucleotides in the reaction chamber are sequentially cleaved and each nucleotide 230 is scanned for labels by STM, AFM, fluorescent microscopy or the use of microfluid channels. The time between labels on labeled nucleotides 220 is recorded. The scanning process is repeated multiple times for single DNA strands to obtain overlapping sets of labeled nucleotide 220 times. Each set of labeled nucleotide 220 time represents the time measurements for a single labeled nucleic acid 230, 240, 250. The multiple monomer-time maps for each DNA strand partially labeled for each nucleotide (see FIG. 1) is collected.

After adjusting for any time difference between labeled and unlabeled nucleotides, a construct is made at 450 for a nucleotide time map 310, 320, 330, 340 for each subsample 110, 120, 130, 140 by combining all of the measured times between labeled nucleotides 220 from similarly labeled strands 230, 240, 250 and aligning the times on a time axis (see FIG. 2.) As noted earlier, the spacing between labels may then be generated. Using an algorithm that assesses/compares the overlap at 420 between all of the measured times, the frequency of signals is generated at 430 and signal analysis at 440 is used to construct a master nucleotide time maps 310, 320, 330, 340, and 350 (see FIGS. 3 and 4 for establishing time/times for adenosine). The computer program should search to find the maximum and most uniform overlap among all of the scanned strands 230, 240, 250 of each subsample of DNA.

The above step is repeated for the other monomers G, C, and T, to complete the unblocked time segments (see FIG. 5). Once all the time segments are filled, a DNA sequence 210 is assembled by aligning 520 the four time maps 310, 320, 330, 340 and eliminating overlap. A computer program may be used to complete this function. When aligning at 520, the time maps 310, 320, 330, 340, it may be useful to find the time map 310, 320, 330, 340 with the greatest number of nucleotides and the map 310, 320, 330, 340 with the second highest number of nucleotides and align 520 those. When aligning 520 the monomer-position maps 310, 320, 330, 340, the non-overlap rule and the rule of minimum sequence 210 length should be utilized.

The computer should find only one possible fit. Next align 520 a third time map 310, 320, 330, 340. Use the non-overlap rule and the rule of minimum sequence 210 length to merge this time map 310, 320, 330, 340 into the previously merged map 310, 320, 330, 340. Do the same thing for the fourth time map 310, 320, 330, 340 to generate a complete nucleic acid sequence 210. Where aligned time maps 310, 320, 330, 340 result in two or more different types of nucleotides located at the same position on the sequence 210, repeat the alignment process using a different alignment. The data analysis is completed when a sequence 210 for the target DNA strand is generated that has no overlapping nucleotides and no gaps in the sequence 210.

All of the compositions, methods and apparatuses disclosed and claimed herein can be made and executed without undue experimentation in light of the present disclosure. While the disclosed compositions, methods and apparatuses have been described in terms of specific embodiments of the disclosed methods and devices, it will be apparent to those of skill in the art that variations may be applied without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the claimed subject matter as defined by the appended claims.

The foregoing detailed description of the preferred embodiments of the disclosed methods and devices has been given for clearness of understanding only, and no unnecessary limitations should be understood therefrom, as modifications will be obvious to those skilled in the art. Variations of the disclosed methods and devices as set forth herein can be made without departing from the scope thereof, and, therefore, only such limitations should be imposed as are indicated by the appended claims.

1. A method of sequencing a polymer comprising:
a) dividing a polymer sample into a number of polymer subsamples equal to a number of different monomer types comprising the polymer sample, wherein only one of the monomer types in each polymer subsample is partially labeled such that an average time between two adjacent labeled monomers is significantly larger than an average time between two adjacent monomers of the same type in the polymer subsample before labelling;
b) sequentially separating each monomer from the polymer subsample;
c) detecting the labels of each separated labeled monomer as a function of time;
d) constructing a time map for each monomer type in each polymer subsample; and
e) assembling the time maps into a polymer sequence.

2. The method of claim 1 wherein the polymer is a nucleic acid, the monomer is a nucleotide, and the number of polymer subsamples and different monomer types is four.

3. The method of claim 2, wherein each subsample comprises from about 1000 to about 100,000 copies of the nucleic acid.

4. The method of claim 2, wherein the labels are bulky groups.

5. The method of claim 4, wherein the bulky groups are selected from the group consisting of organic groups, quantum dots, antibodies, metallic groups and complex organic-inorganic nanoparticles.

6. The method of claim 2, further comprising attaching the labeled nucleic acid strand to a surface.

7. The method of claim 2 wherein sequentially separating each monomer is done by an enzyme and complex organic-inorganic nanoparticles.

8. The method of claim 7 wherein polymer is a nucleic acid and said enzyme has exonuclease activity.

9. The method of claim 1 wherein detecting the time between labels is accomplished/measured with a time-gated detection device.

10. The method of claim 9, wherein the detection device is an optical device, a nanopore device, or an electrical device.

11. The method of claim 1, wherein constructing monomer time maps of each of the polymer subsamples comprises analyzing the measured time by overlapping data analysis and frequency analysis to construct the time maps.

12. The method of claim 1, wherein assembling monomer time maps into a polymer sequence comprises minimum non-overlapping data analysis.

13. A method of sequencing a polymer comprising:
a) dividing a polymer sample into a number of polymer subsamples equal to a number of different monomer types comprising the polymer sample, wherein only one of the monomer types in each polymer subsample is partially labeled such that an average time between two adjacent labeled monomers is significantly larger than an average time between two adjacent monomers of the same type in the polymer subsample before labelling;
b) moving the intact partially labeled polymer across a detector;
c) measuring a time between the partially labeled monomers;
d) constructing a time map for each detected labeled monomer for each partially labeled polymer strand; and
e) assembling the time maps into a sequence for the polymer.

14. The method of claim 13 wherein the polymer is a nucleic acid, the monomer is a nucleotide, and the number of polymer subsamples and different monomer types is four.

15. The method of claim 14, wherein each subsample comprises from about 1000 to about 100,000 copies of the nucleic acid.

16. The method of claim 14, wherein the labels are bulky groups.

17. The method of claim 16, wherein the bulky groups are selected from the group consisting of organic groups, quantum dots, antibodies, metallic groups and complex organic-inorganic nanoparticles.

18. The method of claim 14, further comprising attaching the labeled nucleic acid strand to a surface.

19. The method of claim 13 wherein detecting the time between labels is accomplished/measured with a time-gated detection device.

20. The method of claim 19, wherein the detection device is an optical device, a nanopore device, or an electrical device.

21. The method of claim 20, wherein the detector is -selected from the group consisting of an ion-channel-lipid bilayer sensor, a photodetector, an electrical detector and a mass detector.

22. The method of claim 13, wherein constructing monomer time maps of each of the polymer subsamples comprises analyzing the measured time by overlapping data analysis and frequency analysis to construct the time maps.

23. The method of claim 13, wherein assembling monomer time maps into a polymer sequence comprises minimum non-overlapping data analysis.

24. The method of claim 13, wherein at least one end of each nucleic acid strand is attached to a distinguishable label.

25. An apparatus comprising:
a) a chamber for cleaving a partially-labeled polymer sample into individual monomers;
b) a means for transporting the individual monomers across a detector.

26. The apparatus of claim 25, further comprising: (i) an information processing system; and (ii) a database.

27. The apparatus of claim 25, wherein said detector are capable of detecting labels attached to individual monomers.

28. The apparatus of claim 25, wherein the means for transporting the individual monomers across a detector is a microfluidic chip.

29. The apparatus of claim 25, wherein the detector is selected from the group consisting of an ion-channel-lipid bilayer sensor, a photodetector, an electrical detector and a mass detector.
